(19) United States
(12) Patent Application Publication
(10) Pub. No.: US 2025/0197337 A1
(43) Pub. Date: Jun. 19, 2025

(54) CONVERSION OF TRIACETIC ACID LACTONE TO POTASSIUM SORBATE

(71) Applicant: Wisconsin Alumni Research Foundation, Madison, WI (US)

(72) Inventors: George Huber, Middleton, WI (US); Min Soo Kim, Madison, WI (US); James Dumesic, Verona, WI (US)

(73) Assignee: Wisconsin Alumni Research Foundation, Madison, WI (US)

(21) Appl. No.: 18/540,295

(22) Filed: Dec. 14, 2023

(51) Int. Cl. C07C 51/09

(52) U.S. Cl. CPC C07C 51/09

(57) ABSTRACT

A method to make sorbate salts from triacetic acid lactone. The method includes the steps of hydrogenating triacetic acid lactone to yield 4-hydroxy-6-methyl 2H-pyran-2-one; dehydrating 4-hydroxy-6-methyl 2H-pyran-2-one into parasorbic acid; and ring-opening and hydrolyzing PSA with a base to form a sorbate salt.

### FEDERAL FUNDING STATEMENT

This invention was made with government support awarded under DE-SC0018420 awarded by the US Department of Energy. The government has certain rights in the invention.

### BACKGROUND

The increasing demand for fuels and chemicals produced from sustainable carbon sources has led to a growing interest in using biomass as a renewable feedstock. In particular, 4-hydroxy-6-methyl-2-pyrone (also denoted as triacetic acid lactone, TAL) has emerged as a promising platform chemical that can be converted into a wide range of valuable molecules typically derived from petrochemicals. TAL can be produced synthetically from acetic acid. See Shanks, B. H.; Keeling, P. L., Bioprivileged molecules: creating value from biomass. Green Chemistry 2017, 19 (14), 3177-3185 and Obydennov, D. L.; El-Tantawy, A. I.; Sosnovskikh, V. Y., Triacetic acid lactone as a bioprivileged molecule in organic synthesis. Mendeleev Communications 2019, 29 (1), 1-10. TAL can also be produced via genetically modified biosynthesis routes using natural sources. See Cardenas, et al., Metabolic engineering of Saccharomyces cerevisiae for the production of triacetic acid lactone. Metabolic engineering 2014, 25, 194-203; Sun, et al., Complete and efficient conversion of plant cell wall hemicellulose into high-value bioproducts by engineered yeast. Nature communications 2021, 12 (1), 4975; Liu, et al., Engineering acetyl-CoA metabolic shortcut for eco-friendly production of polyketides triacetic acid lactone in Yarrowia lipolytica. Metabolic Engineering 2019, 56, 60-68; Markham, et al., Rewiring Yarrowia lipolytica toward triacetic acid lactone for materials generation. Proceedings of the National Academy of Sciences 2018, 115 (9), 2096-2101; and Zha, et al., Rational pathway engineering of type I fatty acid synthase allows the biosynthesis of triacetic acid lactone from D-glucose in vivo. Journal of the American Chemical Society 2004, 126 (14), 4534-4535.

Of the commercially valuable end products obtained from TAL, sorbic acid is perhaps the most important. Sorbic acid is used throughout the food and pharmaceutical industries as an antimicrobial preservative. The commercial manufacturing process for sorbic acid involves several steps, including the polymerization of crotonaldehyde and ketene to form an intermediate polyester, which is then decomposed and subjected to various purification steps to yield a high-purity sorbic acid. See, for example, U.S. Pat. Nos. 6,590,122; 6,545,180; 6,525,218; 6,509,498; 6,495,717; 6,462,233 and 6,437,182.

Additionally, sorbic acid has low water solubility, which limits its potential as a food preservative. (Sorbic acid has a solubility of only 0.15 g per 100 ml at room temperature.) In contrast, potassium sorbate (“KS”), a salt of sorbic acid, is more commonly used as a food preservative due to its higher solubility in water (58.5 g per 100 mL at room temperature). As a result, KS is more frequently used due to its higher solubility in water. KS is generally produced by reacting sorbic acid with an equimolar amount of potassium hydroxide (KOH).

The use of TAL-derived sorbic acid as a preservative would be more sustainable than commercial preservatives produced from petroleum resources such as ketene and crotonaldehyde. In this regard, Chia et al. proposed a catalytic upgrading approach to convert TAL to sorbic. See Chia, M.; Schwartz, T. J.; Shanks, B. H.; Dumesic, J. A., Triacetic acid lactone as a potential biorenewable platform chemical. Green Chemistry 2012, 14 (7), 1850-1853. Li and co-workers synthesized other preservatives, such as ethyl sorbate and ethyl benzoate from malonate, crotonaldehyde, and acrolein which can be obtained from biomass. Yuan, L.; Hu, Y.; Guo, X.; Li, G.; Wang, A.; Cong, Y.; Wang, F.; Zhang, T.; Li, N., Biomass-based production of food preservatives. Chem Catalysis 2022, 2 (9), 2302-2311.

### SUMMARY OF THE INVENTION

Disclosed herein is a new method for producing sorbate salts, most notably potassium sorbate (KS) from triacetic acid lactone (“TAL”) The method is a sustainable chemical platform that can be biologically synthesized from natural sources. Sorbic acid and its salts are widely used as a preservative in various foods, pharmaceuticals, and other industrial applications. Instead of making sorbic acid, which is an intermediate for producing the sorbate salts, the method disclosed and claimed herein produces a TAL-derived sorbate salt through three steps, including hydrogenating TAL, converting the resulting 4-hydroxy-6-methyl 2H-pyran-2-one (“HMP”) to parasorbic acid (“PSA”), and ring-opening and hydrolyzing PSA to the sorbate salt. In the preferred version of the method, TAL is fully hydrogenated over Ni/SiO₂ to give near-quantitative yields of HMP. Dehydration of the HMP over a solid acid catalyst yields parasorbic acid (PSA) at roughly 86.5% with respect to TAL. A kinetic model for dehydration of HMP to PSA was developed. When KOH is utilized as a co-reactant in the ring-opening hydrolysis reaction, the reaction results in >99% yield of potassium sorbate (“KS”) from PSA. The TAL-derived KS molecule was analyzed by ¹H NMR and ¹³C NMR. The difference in THF solubility between the TAL-derived KS product and the impurities present allows the TAL-derived KS product to be purified by simply extracting with THF. The purity of KS product so produced and purified was >95.5% by HPLC analysis and ¹H NMR. The overall yield of TAL-derived KS with respect to TAL was calculated to be 79.5%. Finally, the KS formed via the method has the same antifungal activities as commercial KS.

The method yields sorbate salts from triacetic acid lactone as the starting reactant. The method comprises: (a) hydrogenating triacetic acid lactone (“TAL”), for a time, at a temperature, and under conditions wherein at least a portion of the TAL is converted to 5,6-dihydro-4-hydroxy-6-methyl-2H-pyran-2-one (“DHMP”) and/or 4-hydroxy-6-methyl 2H-pyran-2-one (“HMP”); (b) dehydrating at least a portion of the DHMP and/or HMP into parasorbic acid (“PSA”); and (c) ring-opening and hydrolyzing at least a portion of the PSA with a base to form a sorbate salt. The method yields the sorbate salt without producing sorbic acid as an intermediate.

In one version of the method, the TAL is hydrogenated by dissolving it in a solvent to yield a solution and contacting the solution with a hydrogenation catalyst comprising a transition metal. Preferred transition metals include, but are not limited to, nickel, palladium, platinum, and ruthenium and mixtures thereof. It is generally preferred that the transition metal is disposed on a support, such as silica, alumina, titania, and the like. Preferred solvents include tetrahydrofuran (“THF”) and mixtures of THF with water and/or ethanol.

In another specific version of the method, at least a portion of the DHMP and/or HMP is dehydrated to PSA by contacting it with a solid acid catalyst. Preferred solid acid catalyst are selected from the group consisting of heteropoly acids, meso-porous silicas, acid clays, sulfated zirconia, molecular sieve materials, zeolites, acidic material on a thermo-stable support, cross-linked polystyrene containing sulfonic acid groups, and sulfonated tetrafluoroethylene-based fluoropolymer-copolymers.

When the PSA is ring-opened and hydrolyzed by contacting it with KOH, the resulting sorbate salt formed is potassium sorbate. Analogously, when the PSA is ring-opened and hydrolyzed by contacting it with Ca(OH)₂, the resulting sorbate salt formed calcium sorbate. In a preferred version of the method, the PSA is ring-opened and hydrolyzed by dissolving the PSA a solvent comprising water, ethanol, THF, or mixtures thereof, to yield a solution, and adding to the solution a base. Preferred bases include (but are not limited to) NaOH, KOH, and Ca(OH)₂.

### Abbreviations and Definitions

All references to singular characteristics or limitations of the disclosed method shall include the corresponding plural characteristic or limitation, and vice-versa, unless otherwise specified or clearly implied to the contrary by the context in which the reference is made. The indefinite articles “a” and “an” mean “one or more.”

All combinations of method steps disclosed herein can be performed in any order, unless otherwise specified or clearly implied to the contrary by the context in which the referenced combination is made.

The method disclosed herein can comprise, consist of, or consist essentially of the essential elements and steps described herein, as well as any additional or optional ingredients, components, or limitations described herein or otherwise useful in organic chemistry.

CFU=colony-forming units. C-KS=commercial potassium sorbate. DHL=δ-hexalactone. DHMP=5,6-dihydro-4-hydroxy-6-methyl-2H-pyran-2-one. HMP=4-hydroxy-6-methyl 2H-pyran-2-one. KS=potassium sorbate. PSA=parasorbic acid. TAL=triacetic acid lactone. THF=tetrahydrofuran. TSB=tryptic (or trypticase) soy broth. (TSB can be purchased from several international suppliers, including Becton Dickinson GmbH, Heidelberg, Germany; catalog no. 257107.)

The term “solid acid catalyst” is defined broadly herein to include any solid material that has an acid functionality. The solid acid catalysts can comprise one or more solid acid materials. The solid acid catalyst(s) can be used independently or alternatively can be utilized in combination with one or more mineral acid or other types of catalysts. Exemplary solid acid catalysts which can be utilized in the method include, but are not limited to, heteropoly acids, acid resin-type catalysts, meso-porous silicas, acid clays, sulfated zirconia, molecular sieve materials, zeolites, and acidic material on a thermo-stable support. Where an acidic material is provided on a thermo-stable support, the thermo-stable support can include for example, one or more of silica, tin oxide, niobia, zirconia, titania, carbon, alpha-alumina, and the like. The oxides themselves (e.g., ZrO₂, SnO₂, TiO₂, etc.) which may optionally be doped with additional acid groups such as SO4₂— may also be used as solid acid catalysts.

Further examples of suitable solid acid catalysts include strongly acidic ion exchangers such as cross-linked polystyrene containing sulfonic acid groups. For example, the Amberlyst®-brand resins are functionalized styrene-divinylbenzene copolymers with different surface properties and porosities. The functional group is generally of the sulfuric acid type. The Amberlyst®-brand resins are supplied as gellular or macro-reticular spherical beads. (Amberlyst® was once a registered trademark of the Rohm and Hass Company, and is now owned by DDP Specialty Electronic Materials US 8, LLC, Wilmington, Delaware, USA.) Similarly, Nafion®-brand resins are sulfonated tetrafluoroethylene-based fluoropolymer-copolymers which are solid acid catalysts. (Nafion® is a registered trademark of E.I. du Pont de Nemours & Co.)

Zeolites may also be used as solid acid catalysts in the present method. Of these, H-type zeolites are generally preferred, for example zeolites in the mordenite group of fine-pored zeolites such as zeolites X, Y and L, e.g., mordenite, erionite, chabazite, faujasite, etc. Also suitable are ultra-stable zeolites in the faujasite group which have been dealuminated. Suitable zeolites are available from several commercial suppliers, including Clariant AG, Muttenz, Switzerland.

Particularly preferred are the functionalized styrene-divinylbenzene copolymers, exemplified herein by the Amberlyst®-brand resins.

### BRIEF DESCRIPTION OF DRAWINGS

FIGS. 1A, 1B, and 1C. Experimental data and kinetic model for HMP dehydration in THF over Amberlyst® 70-brand resin 100° C. (FIG. 1A), 120° C. (FIG. 1B), and 130° C. (FIG. 1C) in a batch reactor. Reaction conditions: HMP (120.9-135.8 mM) in 40 mL THF solvent, 0.1 g of Amberlyst® 70-brand resin, 30 bar Ar. ●=HMP; ▴=PSA; ▪=Decomposition. Each trace shows the corresponding kinetic model.

FIG. 2. Experimental data and kinetic model for PSA degradation over Amberlyst® 70 resin at 100° C. (●), 120° C. (▴), and 140° C. (▪) in a batch reactor. Reaction conditions: HMP (130.50 mM) in 40 mL THF solvent, 1 g of Amberlyst® 70 resin, 30 bar Ar.

FIG. 3. PSA yield calculated using the kinetic parameters as a function of time at 100° C., 120° C., and 140° C. Reaction conditions: HMP (130 mM) in THF solvent, 0.1 g of Amberlyst® 70 resin, 30 bar Ar.

FIG. 4. HMP conversion to potassium sorbate (KS) in a mixed THF/H₂O solvent. Reaction conditions: Batch reaction, 0.6129 mmol of PSA, 0.6129 mmol of KOH in 20 ml of solvent at 30 bar Ar. ●=130° C. in THF/H₂O (90/10 v/v). ▴=130° C. in THF/H₂O (70/30 v/v). ▪=130° C. in THF/H₂O (25/75 v/v). ♦=100° C. in THF/H₂O (25/75 v/v).

FIG. 5. HMP conversion to KS in EtOH. Reaction conditions: Batch reaction, 0.6129 mmol of PSA, 0.6129 mmol of KOH in 20 mL of solvent at 30 bar Ar. ▪=100° C. ●=130° C.

FIG. 6. Schematic of overall process to make potassium sorbate from TAL as disclosed herein.

FIG. 7. Antibacterial activity of C-KS and TAL-KS against E. coli. Colony forming units (CFU) of log-phase (actively growing) E. coli cultured in TSB liquid media with C-KS and TAL-KS ranging from 1 mg mL to 8 mg/mL. The numbers of live bacterial cells were measured after 24 hours of incubation at 37° C. by counting log CFU/mL. In all cases, 105/ml of log-phase (actively growing) E. coli cells were initially used. Plain TSB medium was used as negative control.

FIG. 8. Antifungal activity of C-KS and TAL-KS at different concentrations (1.5 mg/mL, 2.0 mg/ml, 3.0 mg/mL, 4.0 mg/mL, 6.0 mg/ml and 8.0 mg/mL) against P. chrysogenum. Conidia/mL of P. chrysogenum after 6 days of incubation was measured in log units. In all cases, about 105 conidia/ml of P. chrysogenum was initially used. Plain PDB medium was used as negative control. TAL-derived KS at a concentration of 6 mg/mL showed a significant inhibition of P. chrysogenum growth as compared to commercial potassium sorbate (“C-KS”).

### DETAILED DESCRIPTION OF THE INVENTION

Newly developed and disclosed herein a method for producing potassium sorbate directly from TAL, without passing through sorbic acid as an intermediate. The method, an exemplary version of which is shown in Reaction Scheme 1, yields higher amounts of KS from TAL than previous methods.

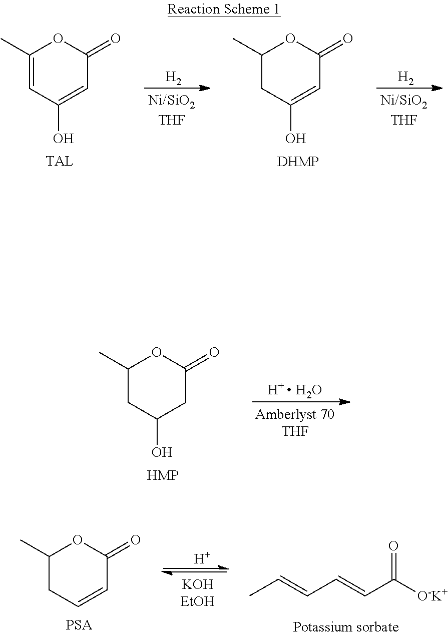

Additionally, the method offers economic benefits as the hydrolysis reaction of sorbic acid to KS is not required. The method yields KS directly from TAL without producing sorbic acid as an intermediate.

To achieve this end, TAL was initially hydrogenated in THE solvent using a Ni catalyst, resulting in a high yield (96.8%) of 4-hydroxy-6-methyltetrahydro-2-pyrone (HMP). Subsequently, the obtained HMP was dehydrated in THE solvent with a solid acid catalyst (i.e., Amberlyst® 70-brand resin) to yield parasorbic acid (PSA). A kinetic model for the dehydration of HMP to PSA was developed. The kinetic model was used to fit experimental data collected. The THF solvent was evaporated and the isolated product containing PSA and by-products was used directly (without further purification) as a feed for ring-opening and hydrolysis to yield KS. To obtain TAL-derived potassium sorbate (TAL-KS), an equal mole of KOH and PSA was added in EtOH solvent, resulting in a quantitative yield of KS from PSA. KOH also successfully served as a co-reactant for the ring-opening hydrolysis reaction. The TAL-derived KS product was obtained as a powder; its purity was >95.5% as measured by HPLC analysis and ¹H NMR after purification using THE extraction. The overall yield of KS from TAL was 79.5%. The TAL-derived KS molecule was characterized using ¹H NMR and ¹³C NMR. Its geometrical configuration was found to be cis-2, trans-4. Commercially available KS is typically the trans-2,trans-4 configuration. Finally, the antimicrobial activity of TAL-KS against bacteria and fungi was demonstrated to be similar to commercial KS.

The first step in the process is to convert TAL to HMP via hydrogenation. This is preferably done using a heterogenous, transition metal-containing or noble-metal containing catalyst. Thus, the hydrogenation catalyst may include one or more metals selected from the group consisting of scandium, titanium, vanadium, chromium, manganese, iron, cobalt, nickel, copper, zinc, yttrium, zirconium, niobium, molybdenum, technetium, ruthenium, rhodium, palladium, silver, cadmium, lanthanum, cerium, praseodymium, neodymium, promethium, samarium, europium, gadolinium, terbium, dysprosium, holmium, erbium, thulium, ytterbium, lutetium, halfnium, tantalum, tungsten, rhenium, osmium, iridium, platinum, gold, mercury, actinium, thorium, protactinium, and uranium. The “noble metals,” which are included within the transition metals, are ruthenium, rhodium, palladium, silver, osmium, iridium, platinum, and gold. Rhenium and mercury are also considered by some to be noble metals.

The catalytic metal is preferably disposed on a support, such as alumina, silica, titania, magnesia, zirconia, etc.

Table 1 lists exemplary catalysts and supports that can be used in the method, along with their conversion rates and selectivity to PSA, 8-hexalactone (“DHL”), 5,6-dihydro-4-hydroxy-6-methyl-2H-pyran-2-one (“DHMP”), and the desired HMP intermediate.

TABLE 1
TAL conversion over different metal catalysts.

| | | | WHSV | Conv. of | Selectivity (%) | | | | |
|---|---|---|---|---|---|---|---|---|---|
| Entry | Catalyst | Conditions | (h⁻¹)^{a} | TAL (%) | PSA | DHL | DHMP | HMP (t/c)^{b} | |
| 1 | Pd/Al₂O₃ | 50° C. and 10.5 h | 2.9 | >99 | 0 | 8.0 | 0 | 92.0 | (71.8) |
| 2 | Pd/Al₂O₃ | 50° C. and 2 h | 15.0 | >99 | 5.2 | 4.9 | 13.0 | 75.1 | (69.2) |
| 3 | Ru/C | 50° C. and 10.5 h | 5.7 | >99 | 0 | 1.4 | 5.1 | 93.7 | (1.8) |
| 4 | Ru/C | 80° C. and 10.5 h | 5.7 | >99 | 0 | 1.0 | 8.7 | 91.8 | (1.6) |
| 5 | Ni/SiO2 | 50° C. and 17 h | 0.3 | 3.3 | 1.6 | 5.0 | 38.2 | 55.2 | (9.7) |
| 6 | Ni/SiO2 | 100° C. and 2 h | 2.3 | 2.8 | 0 | 0 | 70.2 | 29.8 | (3.9) |
| 7 | Pt/SiO2 | 100° C. and 2 h | 30.0 | 0.9 | 0 | 0 | 13.1 | 86.9 | (2.6) |
| 8 | Pt/Si—Al | 100° C. and 2 h | 30.0 | 12.9 | 0 | 7.0 | 62.1 | 31.0 | (4.5) |
| 9 | Ni/SiO2 | 100° C. and 17 h | 0.3 | >99 | 0.3 | 0.4 | 0 | 99.3 | (2.5) |
| 10 | Pt/SiO2 | 100° C. and 17 h | 3.5 | 4.3 | 0 | 0 | 71.3 | 28.7 | (2.8) |

Batch reactions, Mass ratio catalyst: TAL = 1:3, Reaction conditions: 0.1 g of TAL in 20 mL THF, 70 bar H₂,
^{a}WHSV (h⁻¹) = Weight hourly space velocity; g of feed/g of metal/h.
^{b}ratio of trans-HMP to cis-HMP

The hydrogenation of TAL to yield HMP was investigated using various catalysts, including Pd/Al₂O₃, Ru/C, Ni/SiO₂, Pt/Si—Al, and Pt/SiO₂. The conversion of TAL, selectivity towards the desired product, HMP, and other products were studied for different reaction conditions such as temperature, time, and WHSV. From the results, it was observed that Pd/Al₂O₃, Ru/C, and Ni/SiO₂ were able to achieve high conversion of TAL (>99%) with high carbon balance (>95%) under the stated conditions. However, the selectivity towards the desired product, HMP, varied significantly depending on the catalyst and reaction conditions.

The results demonstrated that the Pd/Al₂O₃ catalyst exhibited an activity in the hydrogenation of TAL, achieving a conversion rate of 99% at a reaction temperature of 50° C. and a reaction time of 10.5 hours (Table 1, Entry 1) and 2 hours (Table 1, Entry 2). Moreover, the selectivity towards HMP reached 92% with a remaining selectivity towards the intermediate product, δ-hexalactone of only 8%. Two by-products, PSA and DHL, were also observed in the TAL hydrogenation reaction. PSA was subsequently converted into DHL via hydrogenation. The Ru/C catalyst also exhibited high activity towards TAL hydrogenation, with a conversion rate of 99% achieved at a temperature of 50° C. (Table 1, Entry 3). Although the selectivity towards HMP was higher than that of the Pd/Al₂O₃ catalyst, at 93.7%, the intermediate product, DHMP, still remained, with a selectivity of 5.1% even at a longer reaction time. However, reaction at a temperature of 80° C. and the same reaction time (Table 1, Entry 4), led to a further decrease in the selectivity towards HMP, at 91.8%. The Pt catalyst, on the other hand, showed low activity towards TAL hydrogenation, with a conversion rate of only 0.9%-12.9% achieved.

In the case of the Ni/SiO₂ catalyst, a low conversion rate of only 3.3% was observed at a temperature of 50° C. (Table 1, Entry 5). At a temperature of 100° C. and a reaction time of 2 hours (Table 1, Entry 6), the Ni/SiO₂ catalyst did not form the by-products. At a reaction time of 17 hours and 100° C., with the Ni/SiO₂ catalyst (Table 1, Entry 9), the selectivity towards HMP was the highest, at 99.3%, with DHMP forming at a selectivity of only 0.4%. Notably, the mixture of cis- and trans-HMP was formed while other tested catalysts produced cis-HMP with a negligible amount of trans-HMP. (Data not shown.) On one hand, while the Pd/Al₂O₃ catalyst showed a high HMP production rate, it also produced 8% of selectivity for DHMP. On the other hand, the Ni/SiO₂ catalyst produced fewer by-products, but had a slower HMP production rate.

The results of the initial work on dehydrating HMP to PSA are shown in Table 2. The initial results showed that at a reaction temperature of 140° C., Amberlyst® 70 resin, Amberlyst® 15 resin, and Si—Al did not lead to the formation of sorbic acid. It was also found that the reaction pressure of PSA dehydration over Amberlyst® 70 did not have a significant effect on the PSA yield. See Table 2, Entries 2 and 3. Increasing the reaction time from 2.5 to 8 hours over Amberlyst® 70 resin led to an increase in the conversion of HMP and yield of PSA, but a decrease in the carbon balance. (Table 2, Entries 2 and 5.) Although a slightly higher conversion of HMP was observed with Amberlyst® 15 resin compared to Amberlyst® 70 resin, a lower carbon balance was obtained. See Table 2, Entries 5 and 6.

A longer reaction time of 12 hours at 140° C. resulted in the complete conversion of HMP, but only a yield of 64.2% for PSA was achieved with the reduced carbon balance. (Table 2, Entry 7.) Additionally, no HMP conversion was observed when Si—Al was used at 100° C. (Table 2, Entry 8.) However, a yield of 34.1% for PSA with a 98.9% carbon balance was achieved at a higher temperature of 140° C. and a longer reaction time of 12 hours. (Table 2, Entry 9.) Based on these results, it was hypothesized that decomposition products which are not detected in GC-FID were formed concomitant with increasing the conversion rate of HMP.

TABLE 2
Dehydration of HMP over different acid catalysts

| | | | Conv. of | Carbon | Yield (%) | |
|---|---|---|---|---|---|---|
| | | | HMP | balance | | Sorbic |
| Entry | Catalyst | Condition | (%) | (%) | PSA | acid |
| 1 | Amberlyst 70 | 200° C. and 4 h | >99 | 25.8 | 21.3 | 4.5 |
| 2 | Amberlyst 70 | 100° C. and 2.5 h | 29.1 | 98.3 | 27.4 | 0 |
| 3^{a} | Amberlyst 70 | 100° C. and 2.5 h | 28.4 | 98.1 | 26.5 | 0 |
| 4 | Amberlyst 15 | 100° C. and 2.5 h | 30.7 | 94.4 | 25.0 | 0 |
| 5 | Amberlyst 70 | 100° C. and 8 h | 71.6 | 92.7 | 64.3 | 0 |
| 6 | Amberlyst 15 | 100° C. and 8 h | 78.7 | 85.1 | 67.0 | 0 |
| 7 | Amberlyst 70 | 140° C. and 12 h | >99 | 64.2 | 64.2 | 0 |
| 8 | Si—Al | 100° C. and 2.5 h | 0 | >99 | 0 | 0 |
| 9 | Si—Al | 140° C. and 12 h | 34.1 | >98.9 | 33.7 | 0 |

Batch reactions, Mass ratio catalyst: TAL = 1:2, Reaction conditions: HMP (39.1 mM) in 20 mL THF, 30 bar Ar.
^{a}Reaction pressure of 5 bar Ar

### Kinetic Model for PSA Production Via HMP Dehydration:

Scheme 1 presents the proposed reaction mechanism for the dehydration of HMP. This study assumed that all three reactions in the mechanism are first order and irreversible and that HMP undergoes two parallel reactions represented by equations (1) and (2). The first reaction involves the dehydration of HMP to produce PSA, while the second and third reactions involve the conversion of HMP and PSA into other decomposition products. The concentration of the decomposition products was calculated by the difference between the initial amount of HMP input and the amount of PSA and residual HMP output.

$$HMP {⟶}_{{k}_{3}} PSA + {H}_{2} O \qquad ( 1 )$$

$$HMP {⟶}_{{k}_{3}} {D}_{1} \qquad ( 2 )$$

$$\begin{matrix}PSA {⟶}_{{k}_{3}} PSA + {D}_{2} \\ {D}_{1} , {D}_{2} ≡ decomposition products .\end{matrix} \qquad ( 3 )$$

### Reaction Scheme 2. HMP Dehydration and Decomposition Formation.

The rate expressions for HMP consumption and PSA production can be written as:

$$\frac{d [ HMP ]}{dt} = - {k}_{1} [ HMP ] - {k}_{2} [ HMP ] \qquad ( 4 )$$

$$\frac{d [ PSA ]}{dt} = {k}_{1} [ HMP ] - {k}_{3} [ PSA ] \qquad ( 5 )$$

wherein k=k′[g of Amberlyst® 70]

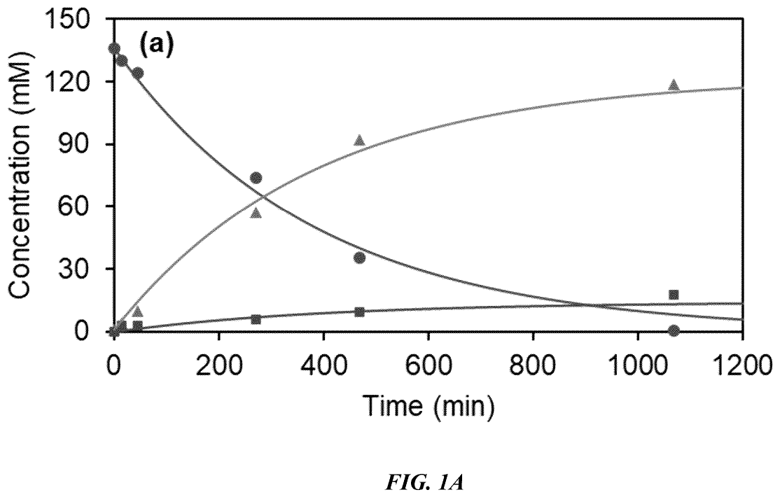

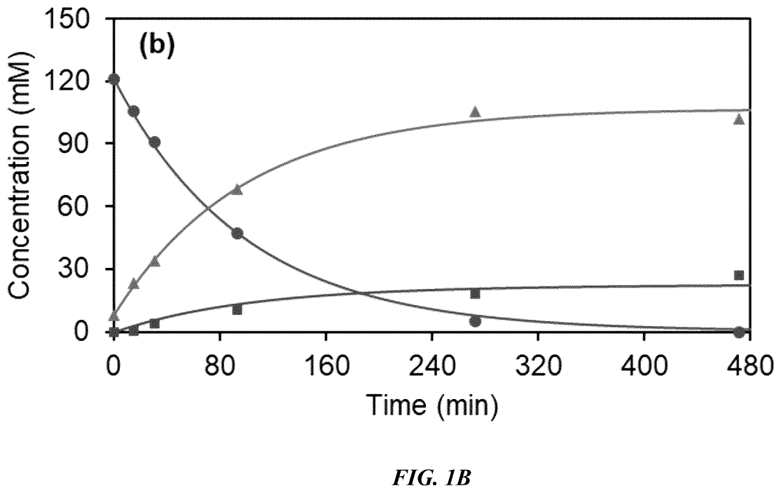

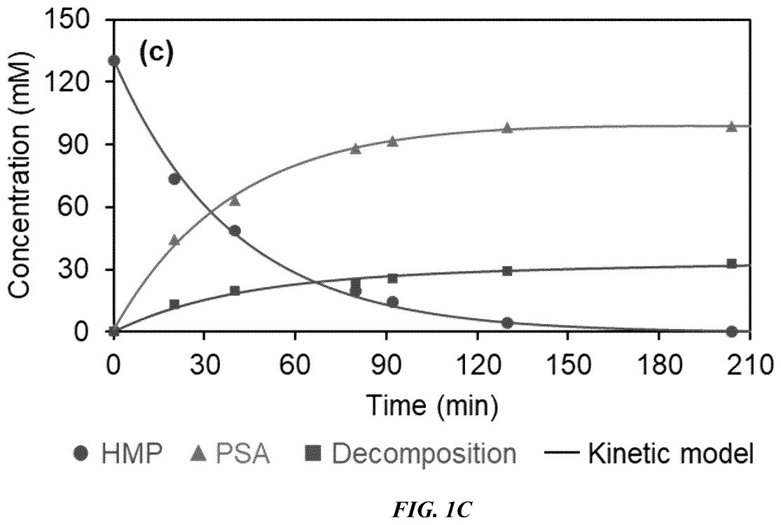

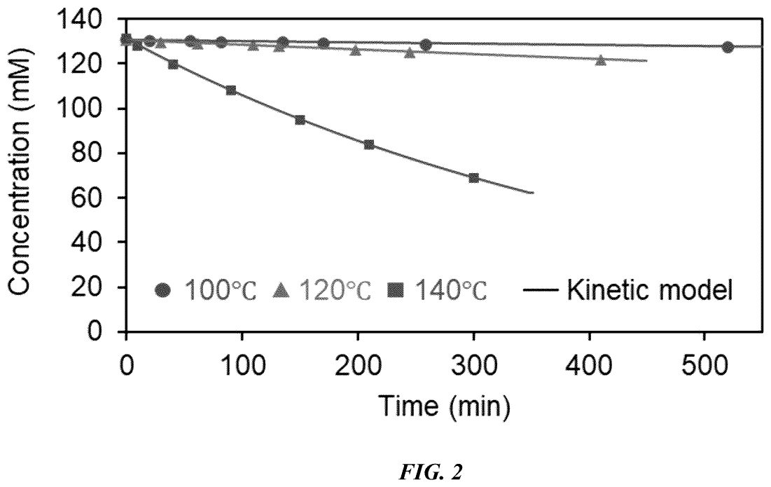

The rate expressions, described in eqn (4) and (5), were used to fit the kinetic data presented in FIGS. 1A, 1B, and 1C. The rate constant of PSA degradation (k₃) presented in FIG. 2 for calculating the rate constants of HMP consumption (k₁) and HMP production (k₂). The disappearance rate was relatively slow. The best correlated values with their standard errors are summarized in Table 3. The activation energy of HMP to PSA was calculated to be 68.6±5.3 KJ/mol. This value differs from previous computational studies, which estimated the activation energies of 78 KJ/mol and 88 KJ/mol for HMP dehydration in THF solvent. (Chia, et al., Mechanistic insights into ring-opening and decarboxylation of 2-pyrones in liquid water and tetrahydrofuran. Journal of the American Chemical Society 2013, 135 (15), 5699-5708. Shrivastav, et al., Elucidating the role of solvents in acid catalyzed dehydration of biorenewable hydroxy-lactones. Reaction Chemistry & Engineering 2020, 5 (4), 651-662.) This discrepancy between the current result and previous simulation studies might be the lack of consideration for the formation of decomposition products in those studies. The experimental results reported here revealed that the activation energy of HMP to PSA was measured to be 74.9±12.6 KJ/mol when considering only the HMP consumption described in eqn (1).

TABLE 3
Estimated Kinetic Parameters for HMP Dehydration
over Amberlyst ® 70 Resin in THF.

| Reaction | Temp. (° C.) | k′ (min⁻¹) | Ea (kJ/mol) | InA |
|---|---|---|---|---|
| HMP dehydration | 100 | 0.0234 | 68.6 ± 5.3 | 18.4 ± 1.6 |
| | 120 | 0.0830 | | |
| | 140 | 0.1983 | | |
| HMP decomposition | 100 | 0.0027 | 97.2 ± 11.4 | 23.4 ± 3.5 |
| | 120 | 0.0180 | | |
| | 140 | 0.0554 | | |
| PSA decomposition | 100 | 0.00004 | 125.0 ± 24.7 | 27.7 ± 7.5 |
| | 120 | 0.00017 | | |
| | 140 | 0.00210 | | |

95% confidence interval in parameter estimation. 1^{st} order rate parameters that are lumped with the amount of catalyst; k = k′[g of Amberlyst ™ 70].

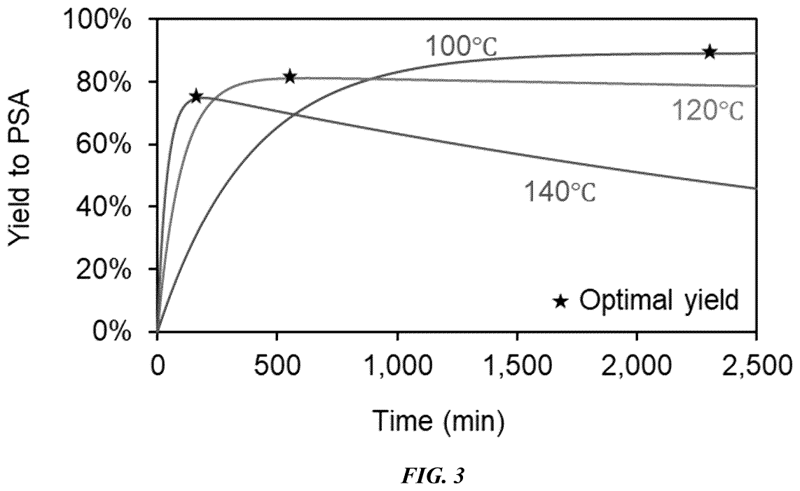

The apparent rate parameters obtained here enabled theoretical calculations of PSA yield in THE over Amberlyst® 70 resin. FIG. 3 illustrates the calculated PSA yields as a function of time at three different temperatures. The results indicate that the optimal yield of PSA occurs at low temperatures and long reaction times because the dehydration of HMP to PSA has the lowest activation energy. The higher yield of PSA was expected at decreased temperatures because the ratios of k₁ to k₃, and k₁ to k₃ decrease with lower temperatures. Based on the theoretical calculations with the kinetic parameters, it was found that the yield of PSA at 100° C. was 88.8%, while at a higher temperature of 140° C. and the same reaction time of 2,440 min, the yield was calculated to be 46.6% due to the degradation of PSA.

### PSA Ring-Opening and Hydrolysis:

To achieve ring-opening and hydrolysis of PSA (to yield KS as the final product), KOH was used. This is notable because the solubility of KOH in THE is limited. To overcome this challenge, a solvent system comprising a mixture of THF and H₂O was employed. To ensure accurate measurements, dimethyl sulfone was added as an internal standard to the PSA feed. The integrated area ratio of dimethyl sulfone (6H) to the PSA (1H) feed in ¹H NMR spectra was expected to be six (6) when the purity of PSA is 100%. In this experiment, 0.1 g of the PSA feed (0.95 mmol) with a purity of 63.2% was mixed with an equal amount of 0.61 mmol of KOH.

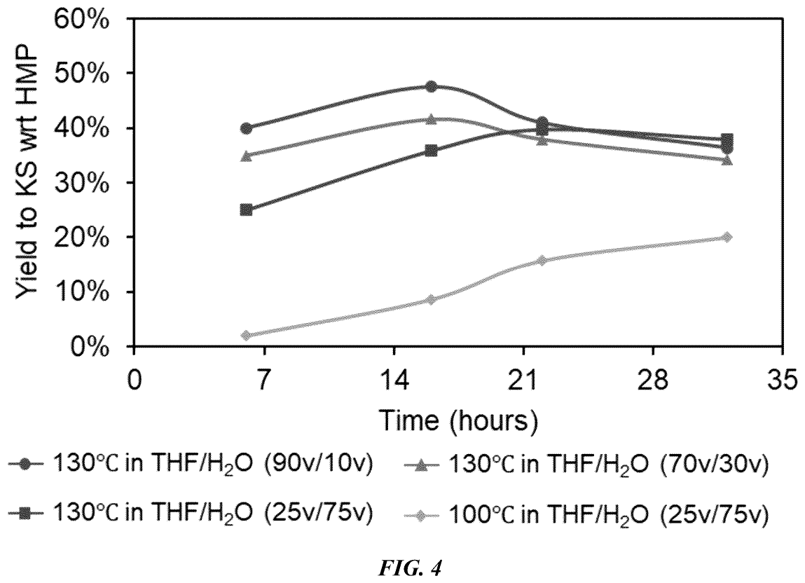

The effect of different THF/H₂O ratios and reaction times on the yield of KS from HMP was investigated. See FIG. 4. At 130° C., the yield of KS from HMP in the mixture of THF and H₂O (90 v/10 v) increased from 40% at 6 hours to 47.6% at 16 hours. However, the yield decreased to 41.0% at 22 hours and further to 36.4% at 32 hours. Similarly, at 130° C., in the mixture of THF and H₂O (70 v/30 v), the yield of KS increased from 35% at 6 hours to 41.7% at 16 hours, then decreased to 38.0% at 22 hours and further to 34.2% at 32 hours. At 130° C. in the mixture of THF and H₂O (25 v/75 v), the yield of KS from HMP was 25% at 6 hours, which increased to 35.8% at 16 hours, then further increased to 39.7% at 22 hours and 38.0% at 32 hours. However, when the temperature was reduced to 100° C. in the same mixture of THF and H₂O (25 v/75 v), the yield of KS from HMP was significantly lower. The yield increased from 2.1% at 6 hours to 8.6% at 16 hours, then increased sharply to 15.8% at 22 hours and further to 20.1% at 32 hours.

It was observed that lower H₂O content resulted in higher yields of KS. These findings highlight the importance of solvent selection in optimizing the hydrolysis process to produce target compounds. Here, EtOH was chosen as the preferred reaction solvent due to its ability to dissolve and disperse KOH, PSA, and KS throughout the reaction medium.

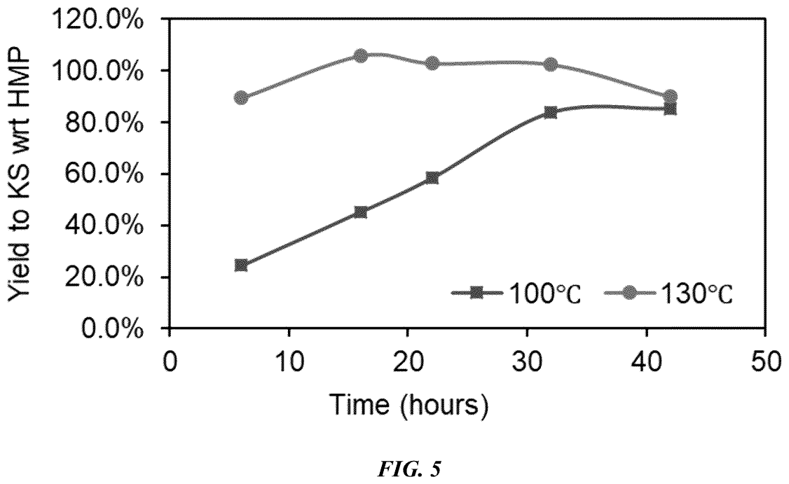

The successful synthesis of KS from HMP was achieved in the EtOH solvent. The effects of temperature and reaction time on the yield of KS from HMP with KOH in EtOH were investigated, as shown in FIG. 5. At 100° C., the yield of KS was relatively low at 24.4% after 6 hours, but increased steadily over time, reaching 85.1% at 42 hours. At a higher reaction temperature of 130° C., the yield of KS was generally higher than at 100° C., but the optimal reaction time was shorter than at 100° C. The highest yield was achieved after 16 hours, at 105.9%, and decreased slightly with increasing reaction time, reaching 89.9% at 42 hours. In the case of a yield over 100%, it was assumed that the yield is greater than 99%. Interestingly, it should be noted that the geometrical configuration of TAL-derived KS was cis-2, trans-4 potassium sorbate, whereas the commercial product is trans-2, trans-4 potassium sorbate. (Data not shown). To determine the configuration of the two isomers, ¹H NMR was used with the reported coupling constants and chemical shift of potassium sorbate isomers The J coupling constants for the cis-2, trans-4 configuration, including J_{H2H3}, J_{H3H4}, and J_{H4H5}, were measured to be 11.4, 11.6, and 14.3 Hz, respectively. In contrast, the J coupling constants for the trans-2, trans-4 configuration were determined to be 15.3, 10.7, and 15.2 Hz, respectively, which agrees with the values reported in the literature. (Cigić, I. K.; Plavec, J.; Možina, S. S.; Zupančič-Kralj, L., Characterisation of sorbate geometrical isomers. Journal of Chromatography A 2001, 905 (1-2), 359-366).

### Overall Process and Purification of TAL-Derived Potassium Sorbate:

The purification of TAL-derived KS was achieved through a simple extraction process using THF, taking advantage of the difference in solubility between KS and impurities in THF. KS has very low solubility in THF, while impurities exhibit high solubility. Consequently, TAL-derived KS remained as a solid while other impurities were dissolved in the THF. After the purification, 6.2 mmol of TAL-derived KS with 95.5% of purity was obtained from 6.6 mmol of the PSA-derived KS having 85.7% of purity. ¹H NMR was used to characterize the TAL-derived KS molecule and the purity of the TAL-KS product was measured by HPLC. Impurities were detected at 1.6 and 3.3-3.5 ppm in ¹H NMR spectra of the raw product which did not appear in the purified TAL-derived KS product. (Data not shown.)

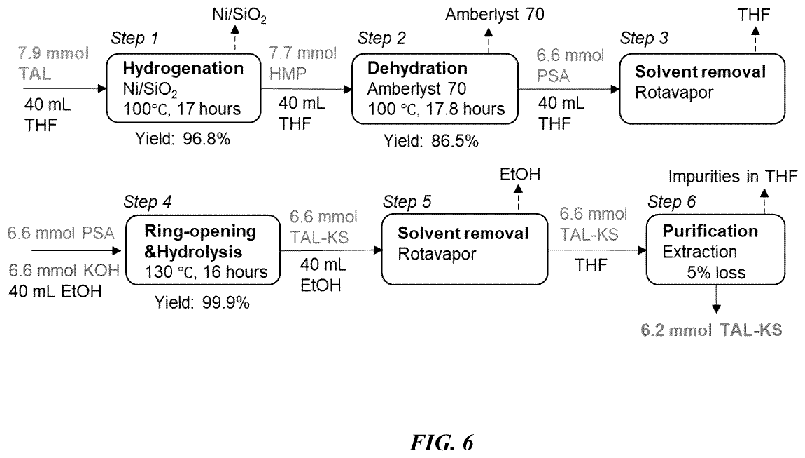

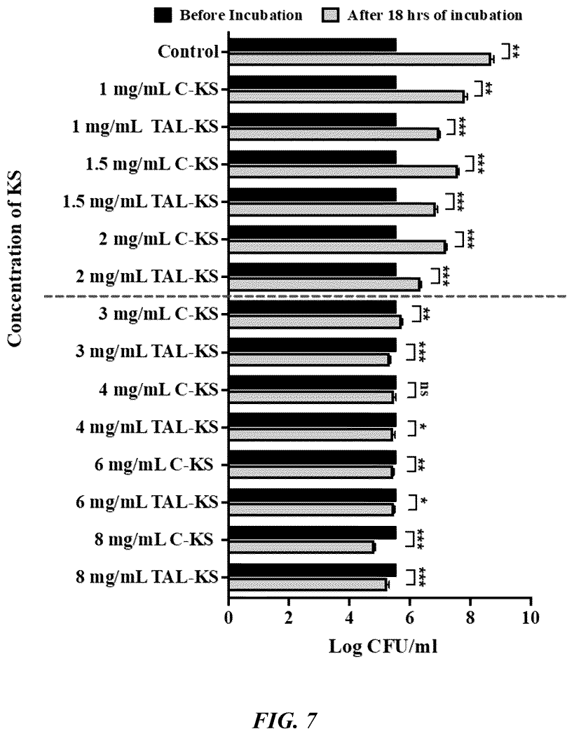

FIG. 6 shows an exemplary version of the overall method of making KS from TAL, based the experimental data reported herein and using the optimal yield for each step. As shown, the method achieves quantitative yields of HMP from TAL and quantitative yields of KS from PSA. The dehydration of HMP to PSA, however, was not as clean, topping out at 86.5% yield of PSA. Despite this, the initial attempts achieved an overall 79.5% yield of KS with respect to TAL. Thus, the method not only affords higher yields to KS than earlier methods, it also realizes economic benefits by eliminating sorbic acid as an intermediate. By not passing through sorbic acid, the present method does not require an additional hydrolysis step to convert sorbic acid to KS. The present method is the first to synthesize potassium sorbate without passing through a sorbic acid intermediate. Thus, the method is a more efficient and economical route to potassium sorbate.

### Antimicrobial Susceptibility Testing of TAL-Derived Potassium Sorbate:

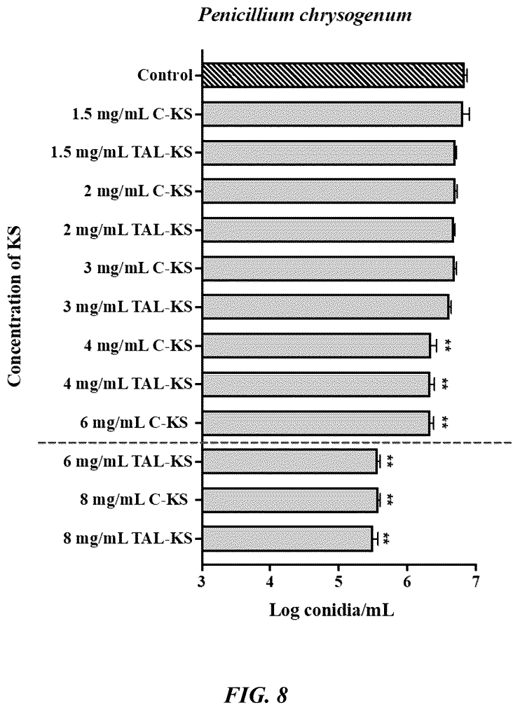

This study evaluated the effectiveness of TAL-derived KS by conducing antimicrobial susceptibility testing against bacteria and fungi. The results were then compared to those of commercial potassium sorbate (C-KS). The minimum inhibitory concentration (MIC) of potassium sorbate against E. coli was determined to investigate its antimicrobial properties. A concentration of 4 mg/ml of both TAL-derived KS and commercial KS had a significant inhibitory effect on the growth of E. coli, while a concentration of 2 mg/ml showed a mild inhibitory effect. (Data not shown.) Therefore, the MIC of both commercial and TAL-derived KS against E. coli was found to be between 2 mg/ml and 4 mg/mL. A separate study showed that the MIC of KS against E. coli O157:H7 (ATCC 35150) was 1.6 mg/mL. (Zhang, et al., Antimicrobial activity and action mechanism of triglycerol monolaurate on common foodborne pathogens. Food Control 2019, 98, 113-119.) Furthermore, another study reported that the MICs of KS against pathogenic strains of E. coli at pH 6.0 ranged from 2.5 to 5.0 mg/mL, which is consistent with the findings of the current study. (Pérez, et al., Effect of pH on the effectiveness of whey protein/glycerol edible films containing potassium sorbate to control non-O157 shiga toxin-producing Escherichia coli in ready-to-eat foods. Food Control 2014, 37, 298-304.) As shown in FIG. 8, both TAL-derived KS and commercial KS at a concentration of >3 mg/ml demonstrated significant inhibitory effects against E. coli.

MIC of KS against Penicillium spp., may vary depending on several factors such as the strain of the fungus, the experimental conditions, and the methods used to determine the MIC. One study found that MIC of KS against Penicillium italicum was 15 mg/ml. (Stanojevic, et al., Antimicrobial effects of sodium benzoate, sodium nitrite and potassium sorbate and their synergistic action in vitro. Bulgarian Journal of Agricultural Science 2009, 15 (4), 307-311.) However, no studies to date have investigated the MIC of KS against P. chrysogenum through MIC testing. The studies performed here found that the MIC of commercial KS was 8 mg/mL, while the MIC of TAL-derived KS was 6 mg/mL. These concentrations were effective in preventing mycelial growth. The data in FIG. 8 consistently demonstrate that spores P. chrysogenum are significantly decreased at concentrations of both TAL-derived KS and commercial KS greater than 4 mg/ml after 6 days of incubation. Notably, a previous study showed that the pure trans, trans-potassium sorbate has a higher antimicrobial activity than the mixture of isomers. (Cigić, et al., supra.) The TAL-derived KS, which is enantiomerically pure cis, trans-potassium sorbate, exhibited similar antimicrobial properties to commercially sourced KS.

Sorbic acid and its potassium salt are already used throughout the food and pharmaceutical industries for the purpose of antimicrobial preservation. Sorbic acid is currently manufactured using a multi-step process that requires the use of petroleum-derived crotonaldehyde and ketene. Potassium sorbate is more frequently used due to its higher solubility in water, but additional steps are required to convert sorbic acid into its potassium salt form. In this regard, using TAL to make potassium sorbate (without making sorbic acid itself) provides an environmentally friendly alternative. The method has three essential steps: hydrogenating TAL, converting the hydrogenated product to parasorbic acid (PSA), and ring-opening and hydrolyzing the PSA to yield KS. It should be mentioned that KOH is served as a co-reactant for ring-opening and hydrolysis reactions of PSA to yield KS. The difference in THE solubility between the TAL-KS product and its impurities allows for facile purification of the TAL-KS product via simple extraction with THF. Analysis of the purified KS product by HPLC analysis and ¹H NMR spectra confirms a high level of purity (>95.5%). The overall yield of TAL-KS is measured to be 79.5%. Interestingly, the geometrical configuration of TAL-KS is cis-2, trans-4 potassium sorbate, whereas the commercial product is trans-2,trans-4 potassium sorbate. As shown herein, the TAL-derived KS and commercial KS possess comparable antimicrobial activities against bacteria and fungi.

### EXAMPLES

The following Examples are included solely to provide a more complete description of the method disclosed and claimed herein. The Examples are not intended to limit the scope of the claims in any fashion.

### Materials:

The following materials were purchased from commercial suppliers: Triacetic acid lactone 98% (“TAL,” Sigma Aldrich, St. Louis, Missouri, USA), 5,6-dihydro-4-hydroxy-6-methyl-2H-pyran-2-one 98% (“DHMP,” Sigma Aldrich), δ-hexalactone 98% (“DHL,” Sigma Aldrich), ethanol 200 proof (“EtOH,” Decon Labs Inc., King of Prussia, Pennsylvania, USA), tetrahydrofuran 99% (“THF,” Alfa Aesar, Ward Hill, Massachusetts, USA), trans, trans-2,4-hexadienoic acid (i.e., sorbic acid, Sigma Aldrich), trans, trans-2,4-hexadienoic acid potassium salt (i.e., potassium sorbate, AmBeed, Inc., Arlington Heights, Illinois, USA). The catalysts used in the Examples were also purchased from commercial suppliers: 64 wt % nickel on silica (Ni/SiO₂, STREM, Newburyport, Massachusetts, USA), 10 wt % palladium on alumina (Pd/Al₂O₃, STREM), 5 wt % ruthenium on carbon (Ru/C, Sigma Aldrich), 5 wt % platinum on silica-alumina (Pd/Si—Al, Riogen, Monmouth Junction, New Jersey, USA), 5 wt % platinum on silica (Pt/SiO₂, Riogen), Amberlyst® 70 (Amberlyst 70, Rohm & Haas, a subsidiary of Dow Chemical, Philadelphia, Pennsylvania, USA), Amberlyst® 15, hydrogen form (Amberlyst 15, Sigma Aldrich), silica-alumina catalyst support, grade 135 (Si—Al, Sigma Aldrich). dimethyl sulfone (Sigma Aldrich), deuterium oxide (D20, Sigma Aldrich), chloroform-d (CDCl₃, Sigma Aldrich). Hydrogenation, dehydration, ring-opening, and hydrolysis reactions were performed in Parr reactor (Parr Instrument Company, Moline, Illinois, USA).

### Product Analysis and Quantification:

### GC-FID Analysis of Products:

A Shimadzu gas chromatograph (GC) equipped with a flame ionization detector (FID) and liquid injection was used to analyze TAL, HMP, DHMP, DHL, sorbic acid, and PSA. A Restek RTX-VMS capillary column having a length of 30 meters, an inner diameter of 0.25 millimeters, and a film thickness of 1.4 micrometers was used. The injection port and FID were maintained at a temperature of 240° C. The sample injection volume was 1 microliter, and a split ratio of 50° C. was employed. The column temperature ramp followed this sequence: 40° C. for 1 minute, then +10° C./min to 180° C., followed by +3° C./min to 240° C. The temperature was held constant at 240° C. for 5 minutes.

### HPLC Analysis of Potassium Sorbate (“KS”):

The degree of purity of KS was measured using high-performance liquid chromatography (HPLC) with a Luna C18 (2) column (Phenomenex Inc., part no. 00G-4252-E0) for separating the samples. The separations were conducted at 50° C. and the detector used was a Waters 2998 PDA, set to 295 nm. The solvent gradient involved a flow rate of 1.0 mL/min of water containing 1% formic acid, which changed linearly to methanol over 20 minutes, followed by 7 minutes of pure methanol, and finally methanol linearly transitioning back to 1% formic acid water during the last 3 minutes. The KS samples synthesized in water at a concentration of 70 mM were compared to commercial KS samples.

### NMR (Nuclear Magnetic Resonance) Characterization of Products:

¹H NMR and ¹³C NMR spectra were obtained by using Brucker Avance-400 spectrometer. Deuterated solvents and dimethyl sulfone (internal standard) were used as references.

### Batch Reactor Studies:

Reactions of TAL and HMP with catalysts were carried out in a 45 mL Parr reactor. The reaction solution and a magnetic stir bar were placed in the reactor. Mechanical stirring was maintained using a magnetic stirrer plate (750 rpm). The reactor was purged three times with 30 bar gas (H₂ or Ar), pressurized to the reaction pressure, then heated to the reaction temperature. The heat-up time was in the range of 15-20 minutes. After reaction, products were cooled to room temperature using an ice bath and filtered with a 0.22 μm PTFE syringe filter prior to analysis.

### TAL and PSA Synthesis:

Because commercially available 4-hydroxy-6-methyltetrahydro-2-pyrone (HMP) and parasorbic acid (PSA) were prohibitively expensive, a scaled-up reaction was conducted to synthesize a larger amount of the compounds. 4 g of TAL was dissolved in 40 mL of THE in a 75 ml Parr reactor with stir bar at 750 rpm and 2 g of Pd/Al₂O₃, TAL/Cat=2:1. The reactor was sealed and purged 3 times with Ar at 30 bar. Then the reactor was then pressurized to 100 bar with H₂. The temperature was kept at 50° C. for the duration of the synthesis (˜18 hours). The heat-up time was in the range of 15-20 minutes. After reaction, the HMP solution was cooled to room temperature using an ice bath and filtered with a 0.22 μm PTFE syringe. HMP was isolated using a rotary evaporator, with the sample heated in a water bath controlled at 40° C. and 50 mbar. 3.8 g of HMP was isolated, corresponding to 96% molar yield with respect to TAL, consistent with experimental results presented in Table 1. PSA was synthesized from the dehydration of HMP over Amberlyst® 70. resin 3 g of the isolated HMP was dissolved in 40 ml of THF in 75 mL Parr reactor with stir bar at 750 rpm and 1.5 g of Amberlyst® 70 resin, HMP/Cat=2:1. The temperature was kept at 140° C. for the duration of the synthesis (˜12 hours). PSA was isolated in the same manner as for HMP. The isolated HMP and PSA were analyzed by 1H and ¹³C NMR (data not shown). The synthesized HMP and PSA were used as the calibration standard for GC analysis.

### KS Synthesis:

The isolated PSA was dissolved in solvents (EtOH or a mixture of THF and H₂O) with the same corresponding mole of KOH to the already known purity of PSA in a 45 mL batch reactor. The reactor was purged three times with 30 bar of Ar, and pressurized to the reaction pressures, then heated to the reaction temperatures. The heat-up time was in the range of 15-20 minutes. After reaction, the KS product solution was cooled to room temperature using an ice bath, filtered with a 0.22 μm PTFE syringe filter. KS was isolated using a rotary evaporator, with the sample heated in a water bath controlled at 40° C. and 50 mbar. The isolated KS remained a solid. The solid KS after evaporation was further dried at 60° C. in a vacuum oven for 12 hours. Finally, the dried solid KS was mixed with the same corresponding mole of dimethyl sulfone (he internal standard for ¹H NMR) to the mole of KOH input in D20, then the yield of KS from PSA was analyzed via 1H NMR and HPLC.

### KS Purification:

The THF solvent extraction was used to purify KS. THF sufficient to cover the sample was added to the isolated KS in a glass vial with a stirring bar. The glass vial was capped to prevent the solvent from evaporating. The vial was mixed in a plate stirrer with 600 rpm stirring at room temperature for 2 min. The glass vial was centrifuged at 2,500 rpm for 10 min to separate KS. The THF solvent was removed. This was repeated three times. Finally, the purified KS was mixed with the same corresponding mole of dimethyl sulfone in D20, then the molecular structure was characterized by ¹H NMR and ¹³C NMR. Purity was measured by ¹H NMR and HPLC.

### Kinetic Modeling:

Experimental data were obtained by inserting a dip tube into the batch reactor. These data were compared to a proposed kinetic model for the HMP dehydration system. The kinetic model consisted of a set of nonlinear ordinary differential equations that were coupled. Rate constants were determined using the Arrhenius equation (which takes into account the temperature dependence of the reaction). Concentration data of reactants and products at different temperatures were used to adjust the rate parameters of the reaction equations numerically. To determine the rate parameters for PSA decomposition over Amberlyst® 70, resin, separate experiments were conducted using 130 mM PSA in THE as feedstock. It was assumed that the decomposition products were not significantly affected by the concentration of HMP and PSA. MATLAB-brand software (v. R2020b, MathWorks, Inc., Natick, Massachusetts, USA) was used to integrate the ordinary differential equations numerically and to estimate the parameters, using the “Isqnonlin” function (solves nonlinear least-squares equation). The “nplarci” function (nonlinear regression parameter confidence intervals) in MATLAB was then used to perform an error analysis of the regressed data, and calculate the 95% confidence interval for each rate constant, using residual and Jacobian matrices. The estimated rate constants were then plotted in an Arrhenius equation, and the pre-exponential factors and activation energies were estimated simultaneously using the LINEST function in Excel, which is a linear least-squares method.

### Preparation of Bacterial and Fungal Strains:

Isolated colonies of Escherichia coli 1-894-1 (ATCC, Manassas, Virginia, USA) were picked from the bacterial culture plate and inoculated into 4-5 mL of TSB liquid medium for all bacterial strains. TSB liquid medium was prepared by dissolving a mixture of 17.0 g pancreatic digest of casein, 3.0 g papaic digest of soybean, 2.5 g dextrose, 5.0 g sodium chloride, and 2.5 g dipotassium phosphate in a final volume of 1,000 mL of distilled water, stirring for at least for 20 minutes, and then sterilizing under high pressure (50 psi for 20 minutes at 121° C.). To achieve log phase growth, bacterial cultures were incubated at 37° C. for 2-6 hours based on the 0.5 McFarland standard, which corresponds to approximately 1.5×108 CFU/mL when the optical density of a bacterial suspension ranged from 0.08 to 0.1. A 1:20 dilution was made by adding 2.0 ml of the original suspension to 38 mL of PBS buffer, and then a 1:10 dilution was prepared by adding 0.01 ml to each well to obtain a final concentration of about 5.0×105 CFU/mL.

To prepare fungal inoculum, Penicillium chrysogenum (ATCC, Manassas, Virginia, USA) was grown on Potato Dextrose Agar (“PDA”) medium (containing 4.0 g potato starch, 20.0 g glucose, and 15.0 g agar in 1 L of distilled water) for 5 days at 25° C. After that, asexual spores (i.e., conidia) were harvested from the PDA medium by using sterile 0.1% Tween-80 solution. The conidia were counted by using a hemocytometer and adjusted to 108 conidia/mL with sterile distilled water. Conidia suspension was stored at 4° C. and used within 2 weeks after the preparation. For long-term freezer storage of bacterial and fungal cultures, an 80% glycerol solution was prepared by diluting 100% glycerol in distilled water. 750 μL of the overnight bacterial cultures and spore suspensions was added to 250 μL of 80% glycerol to make final 20% glycerol suspensions in a 2 mL screw top tube. The prepared cultures were stored at −80° C.

### Antimicrobial Susceptibility Testing:

To assess the effectiveness of KS against bacteria and fungi, several tests were conducted in accordance with the Antimicrobial Susceptibility Testing Standards outlined by the Clinical and Laboratory Standards Institute (Malvern, Pennsylvania, USA) (CLSI Mo2-A12, 2015) with some modifications, including the minimal inhibitory concentration (MIC), and an agar diffusion disc test. Potassium sorbate solutions with concentrations ranging from 0.5 to 8.0 mg/ml were combined with bacterial cells (105 CFU/mL) and fungal spores (105 conidia/mL) in TSB medium with a pH adjusted to 5.5 for bacteria and in Potato Dextrose Broth (“PDB”) medium (with an original pH of 5.5) for fungi. They were then incubated at 37° C. (for E. coli) for 24 hours and 25° C. (for P. chrysogenum) for 6 days. To determine the minimum inhibitory concentration (“MIC”) of antimicrobial substances, optical density (“OD”) at 600 nm was measured through a Bioscreen C device (Oy Growth Curves Ab Ltd., Turku, Finland) using turbidimetric measurements. The MIC refers to the lowest concentration of antimicrobial that prevents visible growth of microorganisms after incubation. For fungal growth, the MIC was determined based on the inhibition of mycelial growth. The negative control was added the same volume of deionized water without potassium sorbate to observe the growth of bacteria and fungi. Furthermore, to determine assess the survivability of cells and spores when exposed to potassium sorbate for 18 hours, microbial counts were expressed as the logarithm of colony forming units per ml (log CFU/mL). Each measurement was performed in triplicate.

An agar diffusion disc test assay was performed to assess the antibacterial and antifungal activities of potassium sorbate. Bacterial suspensions were evenly spread onto trypticase soy agar (“TSA”) plates using a cotton swab, while fungal spore suspensions were spread onto PDA plates, both using the spread plate technique. 6 mm sterile paper disks loaded with 100 μL of KS dissolved in deionized water to make a final concentration of 4 mg and 8 mg per disk. After drying the disks under the fume hood, they were placed on the surface of the TSA and PDA plates, which were then inverted and incubated at 37° C. for 18-22 hours for E. coli, and 25° C. for 6 days for P. chrysogenum to promote growth. Deionized water was used as negative control for both bacterial and fungal strains. Antimicrobial activity was determined by measuring the diameter of the inhibition zone around the 6 mm paper disks.

1. A method to make sorbate salts from triacetic acid lactone, the method comprising:
(a) hydrogenating triacetic acid lactone (“TAL”), for a time, at a temperature, and under conditions wherein at least a portion of the TAL is converted to 5,6-dihydro-4-hydroxy-6-methyl-2H-pyran-2-one (“DHMP”) and/or 4-hydroxy-6-methyl 2H-pyran-2-one (“HMP”);
(b) dehydrating at least a portion of the DHMP and/or HMP into parasorbic acid (“PSA”); and
(c) ring-opening and hydrolyzing at least a portion of the PSA with a base to form a sorbate salt.

2. The method of claim 1, wherein the method yields the sorbate salt without producing sorbic acid as an intermediate.

3. The method of claim 1, wherein in step (a), at least a portion of the TAL is hydrogenated by dissolving it in a solvent to yield a solution and contacting the solution with a hydrogenation catalyst comprising a transition metal.

4. The method of claim 6, wherein the transition metal is selected from the group consisting of nickel, palladium, platinum, and ruthenium.

5. The method of claim 6, wherein the transition metal is disposed on a support.

6. The method of claim 6, wherein the solvent comprises tetrahydrofuran (“THF”).

7. The method of claim 1, wherein in step (b), at least a portion of the DHMP and/or HMP is dehydrated to PSA by contacting it with a solid acid catalyst.

8. The method of claim 7, wherein the solid acid catalyst is selected from the group consisting of heteropoly acids, meso-porous silicas, acid clays, sulfated zirconia, molecular sieve materials, zeolites, acidic material on a thermo-stable support, cross-linked polystyrene containing sulfonic acid groups, and sulfonated tetrafluoroethylene-based fluoropolymer-copolymers.

9. The method of claim 1, wherein in step (c) at least a portion of the PSA is ring-opened and hydrolyzed by contacting it with KOH, whereby the sorbate salt formed is potassium sorbate.

10. The method of claim 1, wherein in step (c) at least a portion of the PSA is ring-opened and hydrolyzed by contacting it with Ca(OH)₂, whereby the sorbate salt formed calcium sorbate.

11. The method of claim 1, wherein in step (c) at least a portion of the PSA is ring-opened and hydrolyzed by dissolving the PSA a solvent comprising water, ethanol, THF, or mixtures thereof, to yield a solution, and adding to the solution a base.

12. The method of claim 11, wherein the base is NaOH, KOH, or Ca(OH)₂.

13. A method to make sorbate salts from triacetic acid lactone, the method comprising:
(a) hydrogenating triacetic acid lactone (“TAL”) by dissolving the TAL in a solvent to yield a solution and contacting the solution with a hydrogenation catalyst comprising a transition metal, for a time and at temperature wherein at least a portion of the TAL is converted to 5,6-dihydro-4-hydroxy-6-methyl-2H-pyran-2-one (“DHMP”) and/or 4-hydroxy-6-methyl 2H-pyran-2-one (“HMP”);
(b) dehydrating at least a portion of the DHMP and/or HMP into parasorbic acid (“PSA”) by contacting the DHMP and/or HMP with a solid acid catalyst; and
(c) ring-opening and hydrolyzing at least a portion of the PSA with a base to form a sorbate salt by dissolving the PSA a solvent comprising water, ethanol, THF, or mixtures thereof, to yield a solution, and adding to the solution a base.

14. The method of claim 13, wherein in step (a) the transition metal is selected from the group consisting of nickel, palladium, platinum, and ruthenium.

15. The method of claim 14, wherein the transition metal is disposed on a support.

16. The method of claim 13, wherein the solvent comprises tetrahydrofuran (“THF”).

17. The method of claim 13, wherein the solid acid catalyst is selected from the group consisting of heteropoly acids, meso-porous silicas, acid clays, sulfated zirconia, molecular sieve materials, zeolites, acidic material on a thermo-stable support, cross-linked polystyrene containing sulfonic acid groups, and sulfonated tetrafluoroethylene-based fluoropolymer-copolymers.

18. The method of claim 13, wherein in step (c) the base is NaOH, KOH, or Ca(OH)₂.
